(19) United States
(12) Patent Application Publication
(10) Pub. No.: US 2013/0338738 A1
(43) Pub. Date: Dec. 19, 2013

(54) DEVICE AND METHOD FOR COGNITIVE ENHANCEMENT OF A USER

(71) Applicants: Gary Nelson Garcia Molina, Eindhoven (NL); Tsvetomira Kirova Tsoneva, Eindhoven (NL); Davide Baldo, Grosseto (IT); Zhu Danhua, Eindhoven (NL); Patrick Hans Arvid Gabrielsson, Hyssna (SE)

(72) Inventors: Gary Nelson Garcia Molina, Eindhoven (NL); Tsvetomira Kirova Tsoneva, Eindhoven (NL); Davide Baldo, Grosseto (IT); Zhu Danhua, Eindhoven (NL); Patrick Hans Arvid Gabrielsson, Hyssna (SE)

(73) Assignee: KONINKLIJKE PHILIPS N.V., EINDHOVEN (NL)

(21) Appl. No.: 13/981,096

(22) PCT Filed: Feb. 28, 2012
(86) PCT No.: PCT/IB2012/050911

§ 371 Date: Jul. 23, 2013

(30) Foreign Application Priority Data
Mar. 2, 2011 (EP) 11156660.0

(51) Int. Cl. A61N 5/06

(52) U.S. Cl. CPC A61N 5/0618
USPC 607/90; 607/88

(57) ABSTRACT

The present invention relates to a device (30) and a corresponding method for cognitive enhancement of a user (21). For effective cognitive enhancement of the user who is going to execute a cognitive activity the proposed device comprises a light unit (31) for providing an imperceptible light stimulation to the user, and a control unit (32) for controlling said light unit (31) to provide said imperceptible light stimulation less than 5 second before the execution of a cognitive activity by the user.

### FIELD OF THE INVENTION

The present invention relates to a device and a corresponding method for cognitive enhancement of a user. Further, the present invention relates to a system for use by a user.

### BACKGROUND OF THE INVENTION

The society has gone through drastic changes over the last century. The lifestyle and the nature of work have evolved and have inevitably brought problems like stress, burnout and mental fatigue. Many people are concerned about the level of stress in their everyday lives. The number of disability claims for burnout has gone up considerably in the past ten years. These problems pose a threat to the physical health and wellbeing of people. The effects spread across various cognitive functions such as attention, memory, perceptual-motor functions, judgment and decision making The high demand for cognitive performance, in particular, is both a reason and a result of stress and burnout. Increased workload, extremely long work hours and intense pressure to perform at peak levels all the time can actually leave people physically and emotionally drained.

These problems have affected almost all ages and professions. Children, adults and elderly are exposed to the demands of the modern society at school, work or home on a daily basis.

### SUMMARY OF THE INVENTION

It is an object of the present invention to provide a device and a corresponding method for effective cognitive enhancement of a user who is going to execute a cognitive activity or task.

In a first aspect of the present invention a device for cognitive enhancement of a user is presented that comprises:

a light unit for providing an imperceptible oscillatory light stimulation to the user, and

a control unit for controlling said light unit to provide said imperceptible light stimulation less than 5 seconds before the execution of a cognitive activity by the user.

In a further aspect of the present invention a corresponding method is presented.

Still further, in an aspect of the present invention a system for use by a user is presented that comprises:

a user device requiring the execution of a cognitive activity by a user and

a device as claimed in claim 1 for cognitive enhancement of the user,

wherein said user device is adapted to provide an information to said device for cognitive enhancement of the user when a cognitive activity is to be executed by the user.

Preferred embodiments of the invention are defined in the dependent claims. It shall be understood that the claimed method and the claimed system has similar and/or identical preferred embodiments as the claimed device and as defined in the dependent claims.

As explained above there is a clear need for cognitive enhancers and this invention is intended in helping people to cope with such a high cognitive demand through (preferably patterned) light that is imperceptible and requires short exposure. The following table summarizes current cognitive enhancing solutions and the respective disadvantages that are addressed by the present invention.

| Known cognitive | |
|---|---|
| enhancer | Disadvantage overcome by this invention |
| Drugs: memory | Side-effects, addiction, tolerance, and adverse |
| enhancers, intelligence | consumer perception |
| boosting drugs, | |
| nicotine, caffeine | |
| Direct brain | Side-effects, surgical intervention and |
| stimulation: cochlear | convalescence |
| implants, retinal | |
| implants, deep | |
| brain stimulation, | |
| trans-cranial | |
| magnetic | |
| stimulation | |
| Software: mental | Long training time is required. There is no clear |
| training software, | evidence that the learned skills are transferred |
| virtual reality | to other cognitive abilities. |
| Neurofeedback | It requires training (lasting for several weeks in |
| | some cases). An important percentage of people |
| | (20%) do not respond to neurofeedback |
| | treatment. |
| Brainwave | Require long exposure periods (~20-30 |
| stimulation | minutes) and regular daily usage to achieve |
| | claimed effect. The frequency of stimulation is |
| | most commonly in the 5-30 Hz range, which |
| | can trigger epileptic seizures and is particularly |
| | annoying. The light stimulation is commonly |
| | delivered via specially designed LED glasses. |
| | This close proximity to the user eyes can pose |
| | safety risks. |

The present invention offers an immediate effect and does not require long stimulation sessions. It ensures user's comfort and safety by using (preferably high frequency) light stimulation and delivering it via a light unit.

The timing of the light stimulation is important according to the present invention. The stimulus is delivered less than 5 seconds, preferably less than 1 second or even only a few hundred milliseconds before the execution of a cognitive activity (or task; both terms are used herein and both meanings shall be understood even if only one of said terms is explicitly mentioned). Further, it is preferred that the stimulus has a repetitive nature in order to make neural circuits in the brain better able to perform a cognitive activity.

In the context of the present invention the term “imperceptible” shall be understood as at most mildly perceivable and unobtrusive in the meaning of non-disturbing and non-interfering with other tasks or activities of the user, i.e. it is not to be understood in the sense that the does not it at all. In case of an oscillatory light stimulation, as preferred in an embodiment, the oscillation is at a rate that is close to the flicker fusion frequency (when the eye cannot longer see any flicker). Preferably, the light stimulation has a frequency in the range from 25 to 200 Hz, in particular in the range from 40 to 80 Hz, or more frequencies in said range.

In an embodiment the control unit is adapted to control frequency, color, intensity, duration, luminance, hue and/or saturation of the imperceptible light stimulation by said light unit so as to enable the adaption of the light stimulation to the user's perception and reaction to light stimulations.

In another embodiment the control unit is adapted to control said light unit to provide one or more further imperceptible light stimulations during and/or after the execution of a cognitive activity by the user, in particular less than 5 seconds after the execution of a cognitive activity by the user (preferably less than 1 second after the execution of a cognitive activity by the user). This may be advantageous for certain applications, e.g. for relaxation after cognitive processing.

Preferably, the control unit is adapted to control said light unit to provide said imperceptible light stimulations for a duration in the range from 100 ms to 5 s, in particular in the range from 300 ms to 3 s to ensure brain entrainment.

Various embodiments exist for the light unit. Preferred embodiments of the light unit are a lamp, a light panel (e.g. a luminescent panel), a screen, a light frame around a device, in particular a screen, or a light therapy device.

Often, a user has a dominant eye which should be stimulated. Accordingly, in an embodiment means for determining the dominant eye of the user are provided. The user may then manually direct the light unit to the determined dominant eye, or alternatively appropriate means are provided for directing the light unit to the determined dominant eye of the user.

Further, in an embodiment monitoring means for monitoring the cognitive engagement of the user are provided, wherein said control unit is adapted to control said light unit based on the monitored cognitive engagement of the user. Thus, the light stimulation can be optimized for the particular user. Preferably said monitoring means comprises means for measuring the brain activity of the user during imperceptible light stimulations, wherein said control unit is adapted to control said light unit based on the measured brain activity.

The present invention can be applied in various systems for use by a user. Such a system may be e-learning system. The system is aware of the activity at hand and can deliver the stimulation at the right moment. In a language learning system the light stimulation is delivered just before the presentation of a word to be learned. Another example is an operation of a complex machinery where the system is aware of the processes that require a particular attention from its operator and timely delivers the stimulation. Alternatively, the system can monitor (using audio-visual information and/or physiological measurements) the cognitive engagement of the user and offer stimulation time coupled to the moments of high cognitive demand.

### BRIEF DESCRIPTION OF THE DRAWINGS

These and other aspects of the invention will be apparent from and elucidated with reference to the embodiment(s) described hereinafter. In the following drawings

FIG. 1 illustrates the influence of constant light on cognition,

FIG. 2 shows a diagram illustrating the influence of oscillatory visual stimulation on brain activity,

FIG. 3 illustrates changes in brain activity (gamma band) following a 3-second long presentation of an RVS at 40 Hz,

FIG. 4 illustrates alpha, theta dynamics for good cognitive performance,

FIG. 5 shows a first embodiment of a device according to the present invention,

FIG. 6 shows diagrams illustrating the reaction time versus the ratio theta (during-task)/theta (pre-task) and versus theta (pre-task),

FIG. 7 illustrates theta and gamma changes during repetitive visual stimulation at high frequency,

FIG. 8 shows diagrams illustrating the reaction time for an experimental group and a control group,

FIG. 9 illustrates the operation of the device according to the present invention,

FIG. 10 shows diagrams illustrating examples of frequency combinations,

FIG. 11 shows various embodiments of a light unit used according to the present invention,

FIG. 12 shows a second embodiment of a device according to the present invention, and

FIG. 13 shows a third embodiment of a device according to the present invention.

### DETAILED DESCRIPTION OF THE INVENTION

Light is necessary for vision and is essential for the execution of various cognitive tasks or activities. In addition to its role as provider of visual information, light can also influence cognitive performance through direct activating effects. To characterize the influence of light exposure on brain activity, two exposure-modalities can be distinguished:
a) Fixed bright light in which a monochromatic light source of constant and high intensity is used;
b) Oscillating light in which some parameter of the light source (usually the intensity) changes over time according to an oscillatory law.

A summary of the presumed mechanisms through which these two light-exposure modalities influence brain activity is presented below to better understand the effect provided by the present invention.

First, the influence of fixed bright light exposure on cognition shall be discussed. It was shown that cognitive performance improves significantly after ˜30 minute-long light exposure both at night and during the day. Such performance-enhancing effects have been shown for a variety of cognitive tasks and activities such as visual search, digit recall, serial addition-subtraction, two-column addition, logical reasoning task, and simple reaction time tasks.

Light-induced modulations of brain activity during engagement in cognitive tasks/activities were detected in numerous brain areas including:

alertness-related structures such as the brainstem,

long-term memory and emotion-related areas such as the hippocampus,

regulation of attention areas such as the dorso-lateral prefrontal cortex, and

working memory related areas such as the middle frontal gyms.

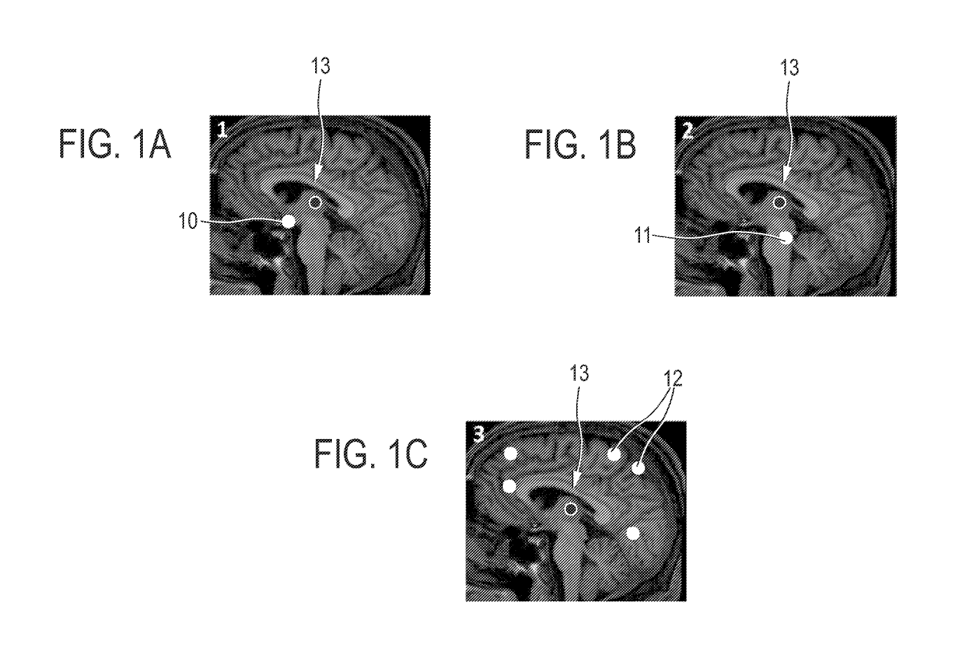

The presumed mechanism through which light influences cognition is illustrated in FIG. 1 (known from G. Vandewalle, P. Maquet, and D.-J. Dijk, “Light as a modulator of cognitive brain function,” Trends in Cognitive Sciences, vol. 13, no. 10, pp. 429-438, 2009). In summary, the irradiance signal reaches cortical sites which are relevant for cognitive functions within 10 to 20 minutes of bright light exposure. FIG. 1A shows the response to light at light onset. The light irradiance signal reaches the hypothalamus indicated by 10. FIG. 1B shows an early response within the first tens of seconds of exposure. After the hypothalamus, the light irradiance signal reaches the brainstem indicated by 11. FIG. 1C shows a late response detected during or after prolonged exposure (16-20 min). The irradiance signal finally triggers a widespread modulation over cortical activity indicated by 12. The thalamic activity is modulated throughout the light exposure indicated by 13.

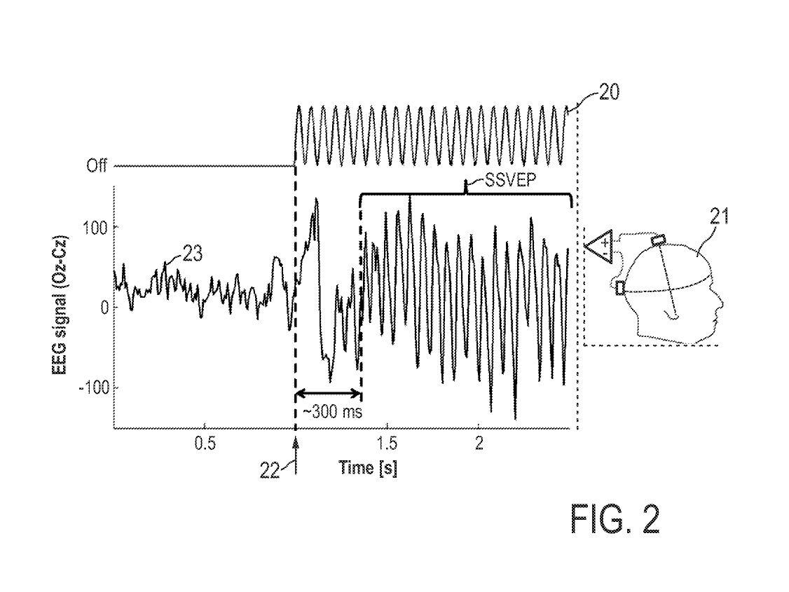

Next, the influence of oscillatory light stimulation on cognition is discussed, in particular the influence on brain activity. Repetitive visual (flicker) stimuli of variable frequency (2-70 Hz) evoke oscillatory responses in the human visual cortex which manifest in the electroencephalogram (EEG) measurements mainly at occipital sites. As illustrated in FIG. 2, a repetitive visual stimulation (RVS) 20 at 15 Hz is presented to the subject 21. A few hundred milliseconds after the onset 22 of (RVS), an oscillatory component in the EEG signal 23 (measured between the occipital position Oz and the center of the head Cz) appears which has the same frequency (and/or harmonics) as that of the RVS 20. The oscillatory response to an RVS is known as Steady State Visual Evoked Potential (SSVEP). The EEG signal 23 particularly shows a sharp voltage increase at the light onset followed by an SSVEP about 300 ms after the stimulus onset 22.

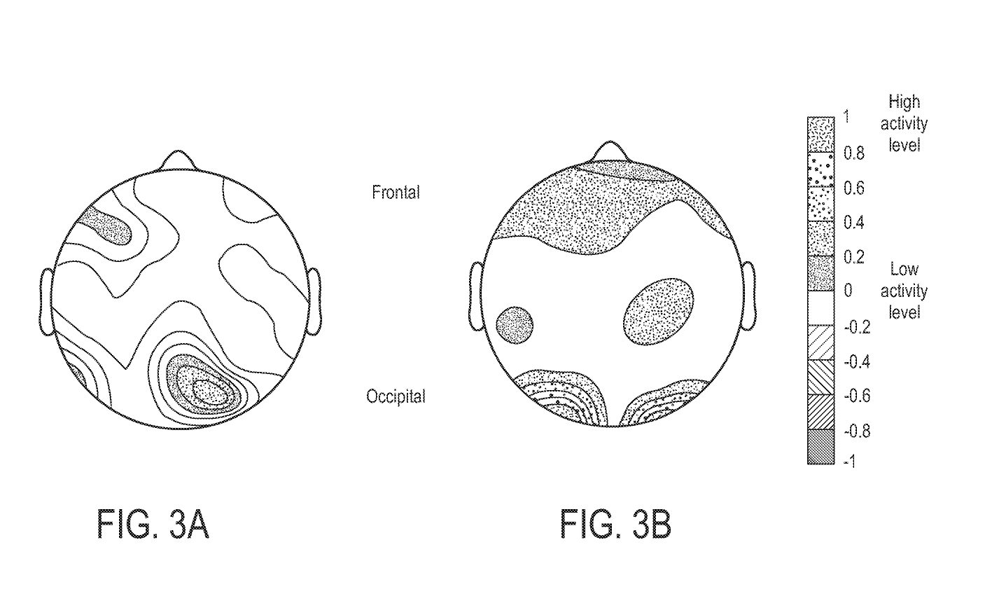

Even though the influence of an RVS prominently appears in the visual cortex, it can also modulate the activity of other cortical sites (as shown in FIG. 3). The RVS influence on the frontal cortex activity is particularly important because this is especially involved in the execution of cognitive functions. FIG. 3 illustrates changes in brain activity (gamma band) following a 3-second long presentation of an RVS at 40 Hz. The RVS was rendered by modulating the on/off sequence of a set of white LEDs which shone through a diffusion screen. The topographic map in FIG. 3A shows a (normalized) representation of the gamma brain activity prior to the stimulation, while the map in FIG. 3B shows the brain activity one second after the stimulation has ended. A clear increase of activity can be observed in occipital sites (visual centers). However, an activity increase is also visible in frontal sites.

Next, electroencephalogram and cognitive processes are discussed. The spectral analysis of EEG signals during different conditions has revealed the existence of rhythms (patterns of activity in defined frequency bands) that characterize behavior as indicated in the following table.

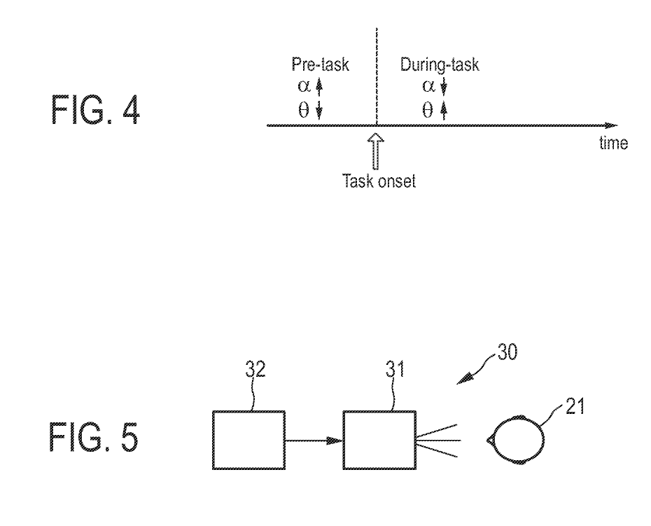

The changes in the EEG during cognitive processing have been extensively studied. A particularly relevant and impactful review (W. Klimesch, “EEG alpha and theta oscillations reflect cognitive and memory performance: a review and analysis”, Brain research. Brain research reviews, vol. 29, no. 2-3, pp. 169-195, 1999) emphasizes the role of alpha and theta rhythms in cognitive performance. According to this review, good performance in a cognitive task is related to i) a pre-task increase in alpha but a decrease in theta power, and ii) a large during-task decrease in alpha but increase in theta. This is illustrated in FIG. 4 showing alpha, theta dynamics for good cognitive performance (according to the above cited review).

Numerous studies reported the coupling between theta and fast (beta, gamma) rhythms during cognitive processing (in particular memory tasks).

Next, the influence on cognition is discussed. Based on the (above mentioned) facts that

EEG rhythms are correlated with cognitive performance, and

repetitive visual stimulation can modulate brain rhythms,

a line of thought has emerged according to which elicitation of the relevant brain rhythm (e.g. alpha, theta for memory) through light can enhance cognitive performance. This approach which is known as brainwave entrainment (BWE) has not provided (up to this date) a scientifically solid proof of its effectiveness. In addition, repetitive visual stimulation at a frequency lower than 30 Hz induces significant visual fatigue, can trigger headaches, and can even cause photo-epileptic seizures. In spite of the risks and absence of solid claim validation, various products which deliver continuous RVS are commercialized.

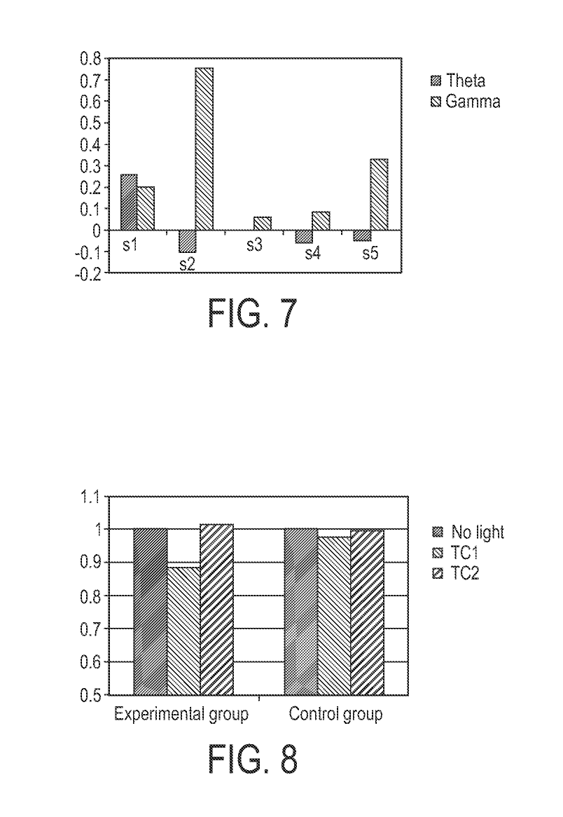

According to the present invention a device 30 (schematically shown in FIG. 5) for cognitive enhancement of a user is provided comprising a light unit 31 for providing an imperceptible light stimulation to the user, and a control unit 32 for controlling said light unit 21 to provide said imperceptible light stimulation less than 5 seconds, in particular less than 1 second, before the execution of a cognitive task by the user. The basic principle of this invention is that light stimulation is delivered prior to the execution of a cognitive task in order to pre-condition brain activity for high performance. A concrete example of this principle comprises lowering the pre-task theta level (see FIG. 4 for explanation) by delivering high frequency repetitive visual stimulation. Indeed, such stimulation lowers the theta level as shown in FIG. 7.

The unique (essential and optional) features characterizing the present invention are reported in the following table.

| Feature | Description | Comparative advantage |
|---|---|---|
| Imperceptible | The light stimulation is | The stimulation is far more comfortable |
| pattern light | rendered through an RVS at | than direct stimulation in the theta (4-8 Hz) |
| stimulation | a frequency in the 40-60 Hz | range. |
| | range. As illustrated in FIG. | This stimulation is safer because the |
| | 7, this stimulation decreases | epileptic seizure risk is considerably lower |
| | theta and consequently | in the 40-60 Hz band. |
| | preconditions the brain | |
| | activity for high | |
| | performance. | |
| Short exposure | Brain activity influencing | The positive effect on cognition is obtained |
| | through repetitive visual | almost immediately. Long exposure times |
| | stimulation occurs within | (such as in the case of bright light) is not |
| | few hundreds of | required. |
| | milliseconds (see FIGS. 2 and | Existing brainwave entrainment devices |
| | 7). | require longer exposure to an RVS. This is |
| | | impractical because the influence on brain |
| | | activity reaches its maximum within one |
| | | second after stimulation onset and |
| | | decreases/increases in an oscillating |
| | | manner. |
| Stimulation | The RVS is applied few | Long exposure to an RVS is bothersome |
| tailored to the | hundred milliseconds before | and potentially unsafe. |
| cognitive task | onset of the cognitive task. | |
| Universal | The RVS is composed of a | The optimum stimulation frequency is user |
| settings | mixture of waves at | dependent. Using the wave mixture it is |
| | frequencies in the 40-60 Hz | ensured that the optimum frequency for a |
| | range. Generally, each | given user is applied. |
| | individual frequency | |
| | modulates brain activity. | |
| User-friendly | The RVS can be rendered by | Changing other light parameters than |
| rendering of the | modulating other light | luminance can bring both, brainwave |
| RVS. | parameters than the | entrainment and user comfort. The comfort |
| | luminance. The hue and/or | aspect is disregarded in existing brainwave |
| | saturation can be changed as | entrainment devices. |
| | well. The brain activity | |
| | entrainment can be achieved | |
| | by alternating between | |
| | colors. | |

The effectiveness and rationale of the present invention have been experimentally tested in a study reported below. First, the validation experiment is described.

Nine subjects were recruited for this study. They were randomly distributed into an experimental group (composed of five subjects S1 to S5) and a control group (composed of four subjects S6 to S9). Each subject participated in three experimental sessions which are described below.

In the first session all subjects were presented with repetitive visual stimuli at all integer frequencies ranging from 40 to 60 Hz. For each subject the optimum frequency (i.e. the one that elicits the highest SSVEP) was determined.

In the second session, all the subjects (in both groups) were requested to perform a “2-back” cognitive test which is used to probe working memory, updating, attention, and sensory processing. This task required participants to attend to a series of letters presented on a computer screen and to state for each letter whether or not it was identical to the letter presented 2 letters earlier. During task performance, the EEG was recorded at 32 locations (sampling frequency 2048 Hz). The process composed of the letter presentation and the corresponding subject's answer is referred to as a trial. A trial is successful when its answer has been correctly given.

The theta dynamics during the time interval spanning from five hundred milliseconds before (pre-task interval) the appearance of the letter to five hundred milliseconds after (during-task interval) the letter appearance have been analyzed. For each subject, the average reaction time (RT) of successful trials versus the ratio

$$\begin{matrix}r = \frac{Energy in the theta band during \text{-} task}{Energy in the theta band p re \text{-} task} \\ = \frac{theta ( during \text{-} task )}{theta ( pre \text{-} task )}\end{matrix}$$

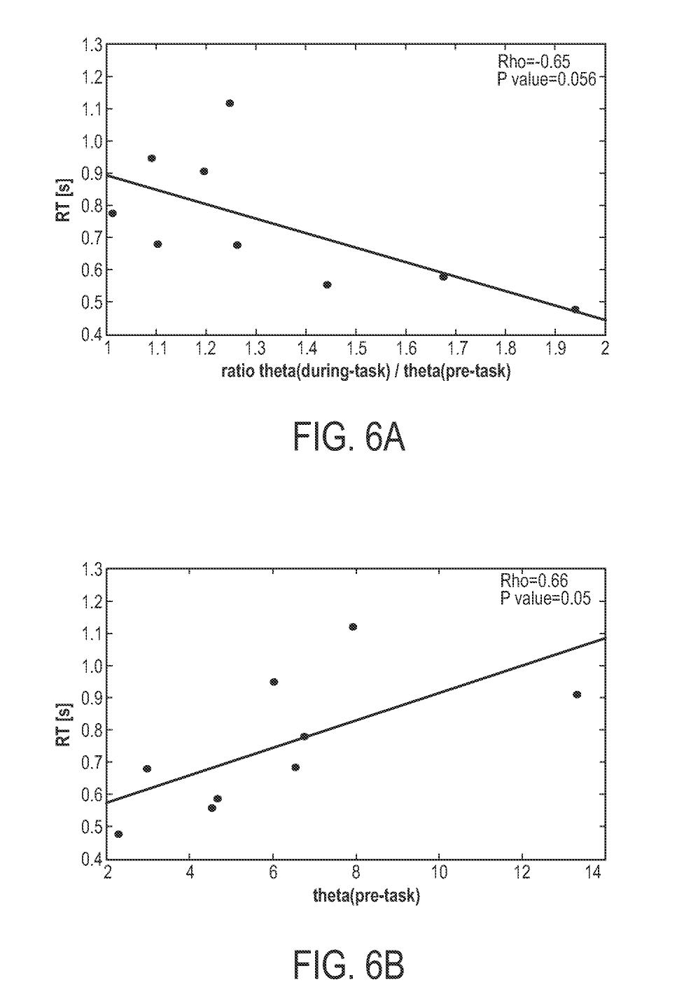

is reported in FIG. 6. Each point in these graphs corresponds to a subject. The correlation coefficient Rho is also reported. FIG. 6A showing the reaction time (RT) versus the ratio theta(during-task)/theta(pre-task) confirms the results reported in the above mentioned review of W. Kliemesch. Indeed, a large ratio r (increase of theta during the task with respect to the level before task) correlates with shorter reaction time. In FIG. 6B showing the RT versus theta (pre-task) it can be seen that the RT shortens when the energy in the theta band, prior to the task, diminishes. This shows that performance can be enhanced by lowering the pre-task theta level.

In the third session subjects in the experimental group were presented with a 700 millisecond-long repetitive visual stimulation (at their individual optimum stimulation frequency) in each trial. Two timing conditions were tested, the first condition consisted in applying the stimulation just before the letter appearance (timing condition 1 or TC1) and the second condition consisted in starting the stimulation simultaneously to the letter presentation (timing condition 2 or TC2). Subjects in the control group were presented with a 700 millisecond-long sham visual stimulation which was rendered through light with random (noisy) intensity variation. The timing conditions were similar to the ones of the experimental group.

First, the gamma and theta dynamics of the experimental group under TC1 are analyzed. The purpose of this analysis is to show that high-frequency RVS increases gamma and decreases theta. FIG. 7 shows the average change in gamma and theta energy levels following light stimulation (repetitive visual stimulation at high frequency) for each subject in the experimental group. The average was made across trials in session 3 and under TC1. A positive value indicates increase due to light stimulation while a negative value indicates a decrease. FIG. 7 reveals that gamma always increases because of light stimulation. For subjects S2 to S5 theta decreases due to light stimulation. For subject S1, theta increases after light stimulation. This special case may need a customized frequency analysis. Indeed, the frequency ranges of brain rhythms need to be tailored to the subject under study.

FIG. 8 represents the average reaction times for the two groups (experimental group on the left and control group on the right) under three conditions: i) no light stimulation (normalized to 1 for convenient comparison), ii) TC1, and iii) TC2. For comparison the average reaction time without light (which was normalized to 1) is also reported. As it can be seen, the reaction time of subjects in the experimental group under TC1 is shorter. This reaction time decrease is more pronounced for the experimental group than for the control group. This positive influence of TC1 on the reaction time is due to the fact that light stimulation preconditions brain activity (by lowering the theta level) for high cognitive performance.

In the following the operation and technical implementations of the device and method according to the present invention will be explained.

Scenarios where the user needs to execute high-level cognitive processes (e.g. memory retrieval, selective attention) at certain moments in time that are externally paced shall be considered first. Examples of such scenarios include:
a) E-learning systems which prompt the user to learn new concepts by presenting them in an audio/visual manner.
b) Foreign language acquisition software that presents users with new words to be memorized.
c) Machine operation which requires special attention from the user at moments where complex/delicate/dangerous tasks are performed. This scenario includes assisted driving, piloting, and simulated navigation.

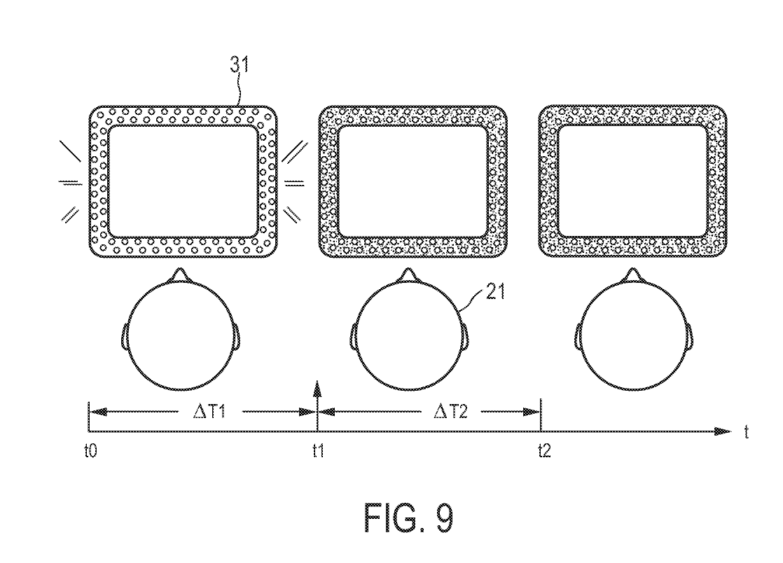

FIG. 9 illustrates the operation principle. In the time interval ΔT1 (from t0 to t1), which is generally less than 5 seconds, preferably less than 1 second, e.g. few hundred milliseconds, before the execution of the relevant cognitive task in time interval ΔT2 (from t1 to t2), light stimulation (rendered using a high frequency RVS by the light unit 31) is applied. This stimulation modulates (pre-conditions) brain activity for high cognitive performance.

As mentioned above, the timing of the light stimulation is important (as shown in FIG. 8). It is important to mention that for different applications such as relaxation after cognitive processing, it can be possible to apply the stimulation during or after the task execution, i.e. during the time interval ΔT2 and/or after time t2.

The properties of the (preferably) repetitive visual stimulation that can be adjusted (by the control unit 32) for this invention are mainly the frequency, light color, intensity, and duration.

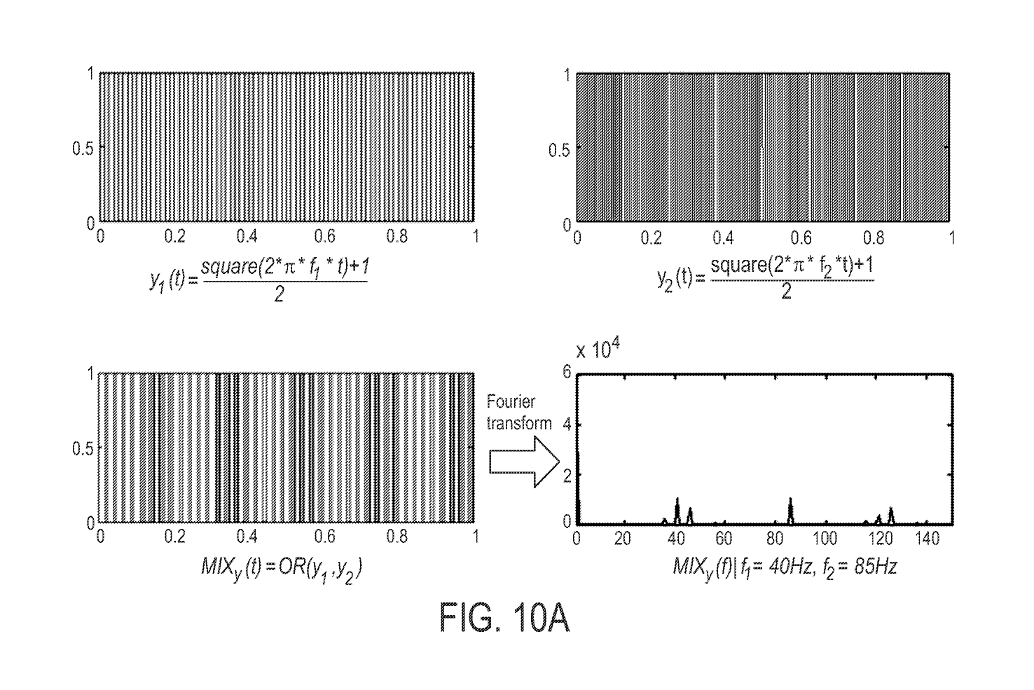

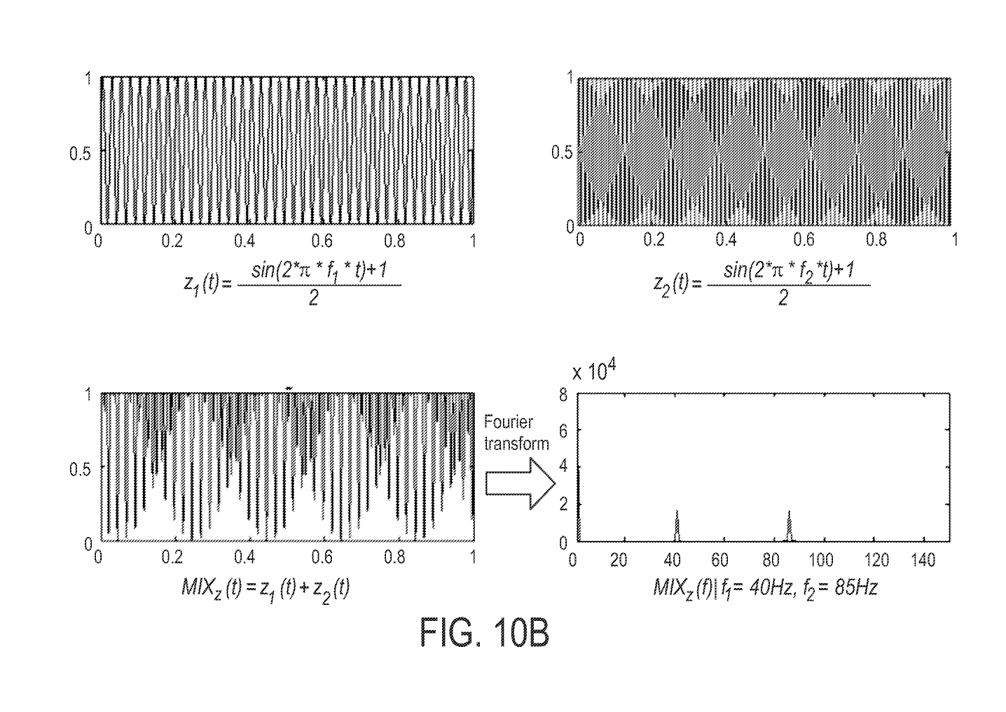

The frequency is the most important stimulation property. Depending on the frequency (or combination of frequencies) of stimulation brainwave entrainment in different frequency bands (theta, alpha, beta, gamma) and, thus, influence of the cognitive performance can be ensured. The optimum stimulation frequency is user dependent. To ensure universality of the stimulation, several frequencies can be combined into a single stimulation device. Indeed, stimulation with a combination of frequencies can ensure entrainment both in the band of the stimulation frequencies, the harmonics, and their linear combinations. FIG. 10 shows an example. In particular, FIG. 10A shows a combination of two square waves (at 40 and 85 Hz) using an “OR” operator and FIG. 10B shows a combination of two sinusoidal waves (at 40 and 85 Hz) using the addition operator.

Another stimulation property is color. Humans have different sensitivity to different colors. Some colors (e.g. blue) have an effect on cognition. Hence, the selection of an appropriate color or combination of colors matters.

The intensity of light can have an effect on circadian rhythm and cognition. Exposure to bright light, compared with dim light, has general central nervous system-activating effects, reduces subjective sleepiness and limits decrements in cognitive performance. Furthermore, the intensity of the light can affect the subjective perception of comfort. Hence, the intensity of the light is carefully chosen to level optimizing the effect on cognition, comfort and safety.

The duration of exposure is another factor that is considered. To ensure brain entrainment the duration of the stimulation is chosen in the interval between 300 ms and 5 s. Generally, the stimulation starts before the execution of the cognitive task and provides quite a short exposure in the range of few hundreds of a second to a few seconds, since for longer exposures habituation of the brain can be observed, i.e. the influence on brain activity diminishes then.

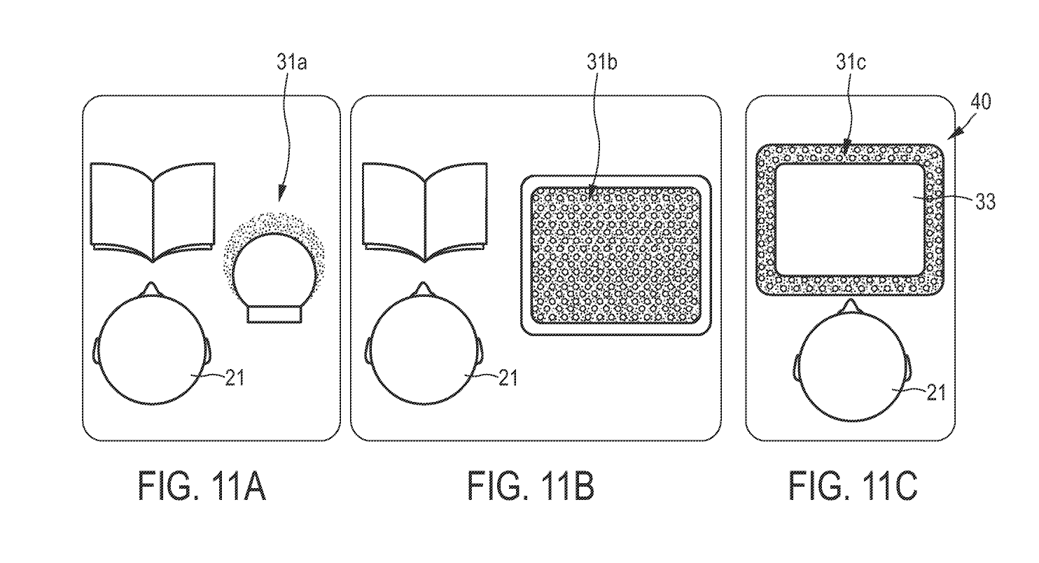

Various embodiment of the light unit 31 are depicted in FIG. 11. The light can be delivered via a dedicated lamp 31a as shown in FIG. 11A, a light panel (e.g. an LED panel or a luminescent panes) 31b as shown in FIG. 11B, or a light frame 31c (e.g. an LED frame or a luminescent frame) around a screen 33 (together e.g. forming a computer monitor 40 or a TV set) as shown in FIG. 11C. Furthermore, light-therapy products can embody such light units. Generally, in any user device requiring the execution of a cognitive task by a user (e.g. a PC, TV set, monitor, light therapy product, table lamp, . . . ) a device 30 can be included.

As mentioned in the above table listing the features of the invention the stimulation can be rendered in a pleasant way by manipulating other light properties such as hue and saturation. For instance, the strength of brain activity modulation can be balanced with the properties of the stimulation. Further, changing the hue or saturation of the light instead of the intensity can also entrain brain activity. The oscillatory light stimulation that is needed can then be rendered by alternating between different hue or saturation levels.

The rendering of the stimulation can also be done using a computer screen by alternating graphic properties of objects in the screen.

Optionally, means for personalization may additionally be provided. The device and method of the proposed invention have settings that make them universal. Additional advantages can be gained if further personalization is carried out. Possible ways to personalize are adaptation to the user's dominant eye and determination of the optimum stimulation properties.

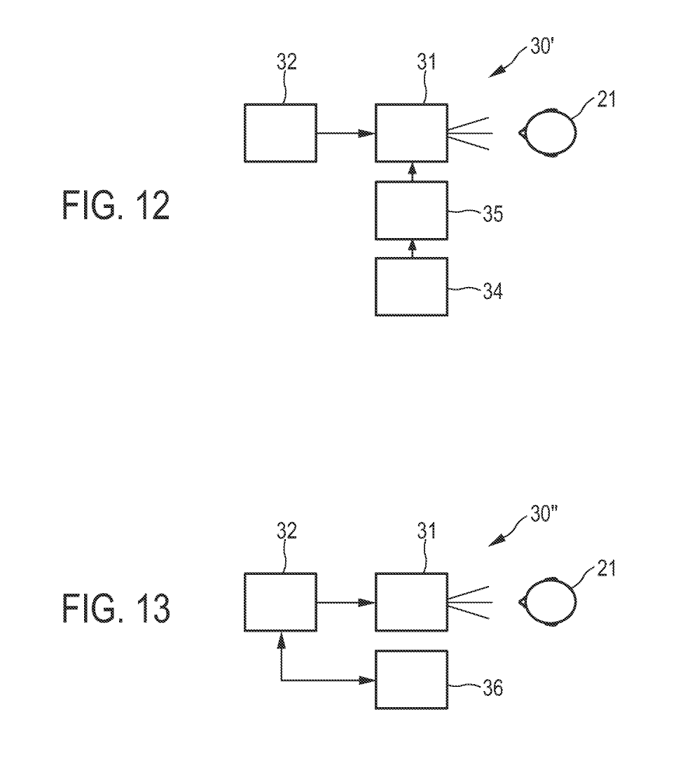

In the case of stimulation using a lamp or a panel, before the start of operation the dominant eye of the person can be identified with a simple test as e.g. described in M. Lasik, “Dominant Eye Test,” http://www.usaeyes.org/lasik/faq/lasik-monovision-dominant-eye.htm. This test can be done separately, e.g. by the user itself, or (as shown in the embodiment of the device 30′ depicted in FIG. 12) appropriate means 34 for determining the dominant eye of the user 21 may be provided. Then, the light unit 31 is placed on the dominant eye side, e.g. manually by the user or automatically by means 35 for directing the light unit to the determined dominant eye of the user (e.g. including mechanical means for moving the light unit 31 in this direction or other means for changing the direction of the light emitted by the light unit 31 appropriately, for instance lenses and/or mirrors).

Further, a short calibration procedure can be done in order to determine the user's specific stimulation properties. During the calibration period (5-10 minutes) the subject is exposed to light stimulation with different properties and his/her brain waves are measured by monitoring means 36 for monitoring the cognitive engagement of the user, (e.g. brain wave measuring means) as shown in FIG. 13 depicting another embodiment of the device 30″. The specific brain response to each type of stimulation is extracted and the stimulation properties ensuring the best brain response are selected, i.e. the light unit 31 is controlled accordingly by the control unit 32 which also controls the monitoring means 36.

For determining the dominant eye of the user a simple test procedure may be used that does not necessarily require any additional devices. A person can do it using its hands or a piece of paper with a hole in the middle. Then the person needs to focus on an object on a certain distance. In an embodiment the means 34 for determining the dominant eye 34 may include a user interface to present some guidelines on and to input the test result and/or to provide an object to focus on.

For directing the light unit to the dominant eye the user can simply move the lamp on the dominant eye side. In an embodiment the means 25 for directing the light unit to the dominant eye of the user may be implemented by a button or software solution to select a stimulation side, e.g. in case of using as a light unit frame around a screen. Further, it is also possible to stimulate from both sides.

The monitoring means 36 for monitoring the cognitive engagement of the user can be implemented by a brainwave sensing device, e.g. a portable headset with EEG sensing capabilities, like the Emotiv (http://www.emotiv.com/) or NeuroSky (http://www.neurosky.com/).

Cognitive enhancement solutions as proposed according to the present invention can be applied to a wide range of user populations and scenarios. Examples include schools to assist students in learning, offices to improve employee performance, and elderly care centers to help patients in slowing down mental ageing. The present invention offers unique advantages compared to known solutions as mentioned in the above tables. Therefore, it is likely to be adopted by a larger consumer size.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims.

In the claims, the word “comprising” does not exclude other elements or steps, and the indefinite article “a” or “an” does not exclude a plurality. A single element or other unit may fulfill the functions of several items recited in the claims. The mere fact that certain measures are recited in mutually different dependent claims does not indicate that a combination of these measures cannot be used to advantage.

Any reference signs in the claims should not be construed as limiting the scope.

1. Device (30, 30′, 30″) for cognitive enhancement of a user comprising:
a light unit (31) for providing an imperceptible light stimulation to the user, and
a control unit (32) for controlling said light unit (31) to provide said imperceptible light stimulation less than 5 second before the execution of a cognitive activity by the user.

2. Device (30, 30′, 30″) as claimed in claim 1,
wherein said control unit (32) is adapted to control said light unit (31) to provide said imperceptible light stimulation less than 1 second, in particular less than 0.5 seconds, before the execution of a cognitive activity by the user.

3. Device (30, 30′, 30″) as claimed in claim 1,
wherein said control unit (32) is adapted to control said light unit (31) to provide an imperceptible patterned light stimulation including a oscillatory pattern.

4. Device (30, 30′, 30″) as claimed in claim 1,
wherein said control unit (32) is adapted to control frequency, color, intensity, duration, luminance, hue and/or saturation of the imperceptible light stimulation by said light unit (31).

5. Device (30, 30′, 30″) as claimed in claim 1,
wherein said control unit (32) is adapted to control said light unit (31) to provide an imperceptible light stimulation having a frequency in the range from 25 to 200 Hz, in particular in the range from 40 to 80 Hz

6. Device (30, 30′, 30″) as claimed in claim 1,
wherein said control unit (32) is adapted to control said light unit (31) to provide an imperceptible light stimulation comprising two or more frequencies, in particular in the range from 25 to 200 Hz, more particularly in the range from 40 to 80 Hz.

7. Device (30, 30′, 30″) as claimed in claim 1,
wherein said control unit (32) is adapted to control said light unit (31) to provide one or more further imperceptible light stimulations during and/or after the execution of a cognitive activity by the user, in particular less than 5 seconds after the execution of a cognitive activity by the user.

8. Device (30, 30′, 30″) as claimed in claim 1,
wherein said control unit (32) is adapted to control said light unit (31) to provide said imperceptible light stimulations for a duration in the range from 100 ms to 5 s, in particular in the range from 300 ms to 3 s.

9. Device (30, 30′, 30″) as claimed in claim 1,
wherein said light unit (31) comprises a lamp, a light panel, a screen, a light frame around a device, in particular a screen, or a light therapy device.

10. Device (30′) as claimed in claim 1,
further comprising means (34) for determining the dominant eye of the user.

11. Device (30′) as claimed in claim 1,
further comprising means (35) for directing the light unit (31) to the determined dominant eye of the user.

12. Device (30″) as claimed in claim 1,
further comprising monitoring means (36) for monitoring the cognitive engagement of the user,
wherein said control unit (32) is adapted to control said light unit (31) based on the monitored cognitive engagement of the user.

13. Device (30″) as claimed in claim 12,
wherein said monitoring means (36) comprises means for measuring the brain activity of the user during imperceptible light stimulations, and wherein said control unit (32) is adapted to control said light unit (31) based on the measured brain activity.

14. Method for cognitive enhancement of a user comprising the steps of:
providing an imperceptible light stimulation to the user, and
controlling said imperceptible light stimulation such that it is provided less than 5 seconds before the execution of a cognitive activity by the user.

15. System for use by a user comprising:
a user device (40) requiring the execution of a cognitive activity by a user and
a device (30, 30′, 30″) as claimed in claim 1 for cognitive enhancement of the user,
wherein said user device (40) is adapted to provide an information to said device (30, 30′, 30″) for cognitive enhancement of the user when a cognitive activity is to be executed by the user.
